(19) United States
(12) Patent Application Publication
(10) Pub. No.: US 2010/0065416 A1
(43) Pub. Date: Mar. 18, 2010

(54) 6-CHLORO-2-TRICHLOROMETHYL PYRIDINE PREPARATION METHOD

(76) Inventor: Feng Dai, Zhejiang (CN)

Correspondence Address: Dr. BANGER SHIA, 102 Lindencrest Ct., Sugar Land, TX, 77479-5201, US

(21) Appl. No.: 12/558,479

(22) Filed: Sep. 11, 2009

(30) Foreign Application Priority Data
Jul. 4, 2008 (CN) 200810062907.5

(51) Int. Cl. C07D 213/61; B01J 19/08

(52) U.S. Cl. 204/157.64; 546/345

(57) ABSTRACT

The present invention discloses a preparation method for 6-chloro-2-trichloromethyl pyridine, in which 2-methyl pyridine hydrochloride or 2-chloromethyl pyridine is used as an initiator and a considerably excess amount of chlorine gas reacts with 2-methyl pyridine hydrochloride or 2-chloromethyl pyridine at a high temperature to form purer 6-chloro-2-trichloromethyl pyridine. The present invention provides a highly selective, high-yield and improved environmental-ly protective method.

### BACKGROUND OF THE INVENTION

(a) Field of the Invention

The present invention concerns a preparation method for 6-chloro-2-trichloromethyl pyridine.

(b) Description of the Prior Art

6-chloro-2-trichloromethyl pyridine is useful as a pharmaceutical and agricultural chemical intermediate, and extraordinary for fertilizer applications. It can be used as an improved nitrogenous fertilizer for agriculture that causes a delay in the nitration of NH₄⁺ and improves soils and plant nutrition. Therefore, it is desired to find an efficient preparation method. Previous methods for preparing mixtures rich in 6-chloro-2-trichloromethyl pyridine have been reported in U.S. Pat. No. 3,424,754, entitled “Process for 2-chloro-6-(trichloromethyl) pyridine composition” and U.S. Pat. No. 3,420,833, entitled “Vapor phase production of polychlorinated compounds”. In the method of U.S. Pat. No. 3,424,754, chlorine gas is introduced into a liquid system containing 2-methyl pyridine hydrochloride, and the reaction takes place at 200° C. to obtain 75% volatiles of mixtures containing 90% 6-chloro-2-trichloromethyl pyridine in a net yield of 68% of 6-chloro-2-trichloromethyl pyridine. The HCl generated during the reaction reacts with the 2-methyl pyridine in another vessel to form liquid methyl pyridine hydrochloride. The weight ratio of Cl₂ to 2-methyl pyridine in the reaction as described in U.S. Pat. No. 3,424,754 is 1:1 or 2:1. Although the reaction can be carried out, the thus obtained mixture contains a large amount of tar and polymers that are difficult to be processed and separated. U.S. Pat. No. 3,420,833 describes the production of mixtures rich in 6-chloro-2-trichloromethyl pyridine by reaction of 2-methyl pyridine vapor with chlorine at 400° C. in the presence of a diluent. Such a process is quite energy consuming because all feeds and diluents must be vaporized. U.S. Pat. No. 3,418,323, entitled “2-chloro-6-(trichloro-methyl)pyridine compounds” describes the preparation of 6-chloro-2-trichloromethyl pyridine by reacting chlorine with 2-trichloromethyl pyridine in a liquid phase at 120-135° C. in the presence of ultraviolet light. Only small amount of 6-chloro-2-trichloromethyl pyridine can be obtained but 3,5-dichloro-2-trichloromethyl pyridine and 3,6-dichloro-2-trichloromethyl pyridine are formed in a large amount by means of such a method. The selectivity of the reaction is low. The mixtures obtained by these methods as mentioned in the previous literatures are difficult to be processed. The method described in U.S. Pat. No. 4,577,027, entitled “Production of polychlorinated pyridine mixtures by direct liquid phase chlorination of alpha-picoline” is different from those in the previous literatures. However, U.S. Pat. No. 3,420,833 describes the production of mixtures rich in 6-chloro-2-trichloromethyl pyridine by reaction of chlorine with 2-methyl pyridine at 100-250° C. in the presence of a CCl₄ diluent. Such a process is impractical and produces many by-products. Great losses of the diluent, CCl₄, would pose a significant hazard to the atmospheric environment. Moreover, CCl₄ is forbidden to be produced and used by A.D. 2010, and thus makes it difficult to be commercialized.

### SUMMARY OF THE INVENTION

It is an object of the present invention to provide a preparation method for 6-chloro-2-trichloromethyl pyridine. The present invention mainly relates to a preparation method in which 2-methyl pyridine hydro-chloride or 2-chloromethyl pyridine is used as an initiator, and a considerably excess amount of chlorine gas reacts with 2-methyl pyridine hydrochloride or 2-chloro-methyl pyridine at a high temperature to form purer 6-chloro-2-trichloromethyl pyridine, and this method possesses features of high selectivity, high yield, better environmental protection, economy and benefit to commercial production.

To achieve the foregoing object, the present invention provides the following technical solution:

The preparation method for 6-chloro-2-trichloro-methyl pyridine comprises using gaseous chlorine and 2-methyl pyridine as raw materials, firstly, feeding an initiator into a first reactor, continuously introducing a considerably excess amount of gaseous chlorine thereinto, reacting the gaseous chlorine with the initiator to form HCl gas at 140° C.-230° C., preferably at 190° C.-210° C., the HCl gas rising and entering a second reactor, forming 2-methyl pyridine hydrochloride under controlled temperature conditions in counter-current flow of the raw material, 2-methyl pyridine, the hydrochloride re-entering the first reactor with a liquid phase system containing the initiator, reacting with the excess gaseous chlorine to form 2-trichloromethyl pyridine followed by further reacting with the continuously introduced excess gaseous chlorine to form a volatile mixture rich in 6-chloro-2-trichloromethyl pyridine until a stable state is reached, withdrawing the mixture from the bottom of the first reactor, and then purifying the mixture to obtain a 6-chloro-2-tri-chloromethyl pyridine compound, and the second reactor is connected back to a tail gas recovery unit, and the reaction equation is:

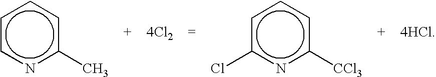

The reaction in said preparation method is a continuous reaction or an intermittent reaction; said purification is performed by rectification in a plate column or recrystallization with ethanol. The materials can be fed to the middle of a column having 35 theoretical plates, with a rectifying section having 20 theoretical plates, and a stripping section having 15 theoretical plates. A small amount of a low boiling volatile material (2-trichloromethyl pyridine) is distilled off at the top of the column, and the low-boiling material is returned to the first reactor for chlorination and continues to react to form 6-chloro-2-trichloromethyl pyridine. The materials in the column are continuously pumped and fed to the middle of a second rectification column having 35 theoretical plates. A light-component product at the top of the second rectification column is very pure 6-chloro-2-trichloromethyl pyridine, which is obtained as a white crystalline solid after cooling. A heavy component in the column is 3,6-dichloro-2-trichloromethyl pyridine, which can serve as an intermediate to produce herbicides. Furthermore, purer 6-chloro-2-trichloromethyl pyridine can obtained by recrystallization with ethanol, that is, the chlorinated product can be recrystallized with ethanol in a weight:volume ratio of 1:1. The content of resulted volatile purer 6-chloro-2-trichloromethyl pyridine determined using GC is 98%. The ethanol can be repeatedly used, and the distillation residue obtained after the purification can be repeatedly used as the initiator for the chlorination in the first reactor. The present method is superior to the method described in U.S. Pat. No. 3,424,754, entitled “Process for 2-chloro-6-(trichloromethyl) pyridine composition”, in which the product is crystallized from pentane/dichloromethane. Pentane and dichloromethane have relatively low boiling points, and pentane also has a low flash point. Therefore, this results in a higher solvent consumption and causes a more significant safety and environmental risk.

The weight ratio of said raw materials of gaseous chlorine to 2-methyl pyridine is from 4:1 to 20:1. Relative to the 2-methyl pyridine, a considerably excess amount of the chlorine gas is required for the chlorination. This can provide additional agitation, a better mixing effect and a higher partial pressure of the chlorine gas. The higher partial pressure of the chlorine gas increases the solubility of the chlorine gas in the reaction medium.

Said initiator is a homogeneous solution of liquid 2-methyl pyridine hydrochloride or 2-chloromethyl pyridine, i.e., a liquid phase system comprised of 2-methyl pyridine hydrochloride or 2-chloromethyl pyridine that is not dissociated from the solid. Generally speaking, 2-methyl pyridine hydrochloride or 2-chloromethyl pyridine is solid, but is liquid in the presence of excess HCl gas at a positive temperature.

Said certain temperature in the second reactor is the salt forming temperature in the second reactor, which is controlled at 20-70° C., preferably 50° C. The generated HCl gas enters the second reactor, which needs to be partially condensed using tap water as cooling water, such that the excess HCl gas reacts with the downwardly flowing 2-methyl pyridine under controlled temperature conditions to form liquid 2-methyl pyridine hydrochloride. The condenser carries away the large amount of heat generated during the formation of the hydrochloride, but still maintains a positive temperature to allow the downwardly flowing 2-methyl pyridine hydrochloride to be in a liquid state and not oxidized by the chlorine gas. In the case where the temperature in the second reactor is lower than 20° C., the solid hydrochloride would be formed. Entry of the solid hydrochloride into the liquid system in the first reactor would induce polymerization and decomposition reactions. If the temperature is above 70° C., the product will be chlorinated into tarry matter. Entry of the tarry matter into the first reactor would cause decomposition of the chlorinated liquid system. So, maintaining the temperature at 50° C. during the salt formation is more stable.

Said certain temperature used in the reaction is the reactant temperature, which is controlled at 140-230° C., and most preferably within a temperature range of 190-210° C. The minimum weight ratio of chlorine gas to 2-methyl-pyridine is 4:1. As the temperature is elevated above 180° C., the weight ratio of chlorine gas to 2-methyl pyridine must increase in order to achieve a high yield of desired volatile 6-chloro-2-trichloromethyl pyridine. As the temperature is elevated, chlorine gas reacts more rapidly with 2-methyl pyridine, so it requires a faster supply of chlorine gas to the reactor. An increase in the weight ratio of chlorine gas to 2-methyl pyridine can thus realize a higher partial pressure of the chlorine gas and a greater mole fraction of chlorine in the reaction medium. While the solubility of chlorine gas decreases as the temperature rises, the rise in system pressure results in a higher solubility of chlorine gas. Generally speaking, if the reaction takes place above 190° C. in a Cl₂ to 2-methyl pyridine weight ratio of less than 4:1, the reaction liquid system is carbonized. Therefore, the Cl₂ to 2-methyl pyridine weight ratio of 1:1 or 2:1 described in U.S. Pat. No. 3,424,754, entitled “Process for 2-chloro-6-(trichloromethyl) pyridine composition” is impractical. The reaction can take place, but the finally obtained mixture is mixed up with many carbides and is difficult to be processed.

When the continuous reaction is used in said preparation method, the bottom of the first reactor is connected in series to the other chlorination reactor. When the chlorination reaction in the first reactor reaches a stable state, the product is continuously withdrawn from the bottom of the first reactor and enters the other chlorination reactor connected in series thereto, where the reaction is carried out at 125° C.-230° C. with the continuous introduction of chlorine gas and without the introduction of 2-methyl pyridine. The reaction velocity increases with rising temperature. The reaction can be carried out under irradiation with ultraviolet light or under no irradiation, preferably under irradiation with ultraviolet light. 2-trichloromethyl pyridine generated in the stable state is converted into 6-chloro-2-trichloro-methyl pyridine followed by the purification. When the intermittent reaction is used, the reaction is stopped and 2-methyl pyridine is added dropwise into the first reactor when the reaction in the first reactor reaches a stable state. Subsequently, 2-trichloromethyl pyridine is converted into a mixture rich in 6-chloro-2-trichloro-methyl pyridine directly under irradiation with ultraviolet light, with the continuous introduction of chlorine gas, followed by the purification.

Said stable state is a state when the proportion of each component in the product does not significantly vary any more after the overall reaction takes place for a certain time. The time taken to reach a stable state is associated with the reaction temperature, the ratio of chlorine gas to 2-methyl pyridine added, and the volume of the reactor. The lower the reaction temperature, the longer the time required to reach a stable state. Generally speaking, in a 500 L reactor, it takes 17 hours to reach a stable state in a chlorine gas to 2-methyl pyridine added weight ratio of 10:1 at a reaction temperature of 195° C.

The reaction can be carried out between 140° C. and 230° C. When the reaction temperature is between 140° C. and 190° C., a significant amount of 2-trichloromethyl pyridine and 3,6-dichloro-2-trichloromethyl pyridine is formed. The lower the temperature, the more the amount of both compounds. At a temperature lower than 140° C., 6-chloro-2-trichloromethyl pyridine cannot be obtained. From further research, it was found that the amount of both 2-trichloro-methyl pyridine and 3,6-dichloro-2-trichloromethyl pyridine reduces when the temperature reaches 190° C.-230° C. Thus, very pure 6-chloro-2-trichloromethyl pyridine can be obtained. When the temperature exceeds 230° C., a large amount of carbonized materials are formed, resulting in a mixture that is difficult to be processed.

Said tail gas recovery unit in turn comprises the connection of a first heat exchanger, an intermediate receiving drum, a second heat exchanger, a tail gas separator, a chlorine gas dryer and a gas mixer for recovering excess tail gas from the reaction, namely, the gaseous chlorine and HCl gas, together with a mixture of partially volatile 6-chloro-2-trichloromethyl pyridine and 2-trichloromethyl pyridine, which is carried by the tail gas and rises and enters the second reactor.

After said mixture of partially volatile 6-chloro-2-trichloromethyl pyridine and 2-trichloromethyl pyridine passes through the second reactor and the first heat exchanger, the major part thereof is collected back to the first reactor. A small amount of the volatile material enters the intermediate receiving drum and the second heat exchanger and then is directly recovered into the first reactor through the bottom of the intermediate receiving drum. The gaseous chlorine and HCl gas are treated in the tail gas separator, subjected to falling film absorption and spray absorption, wherein the HCl gas is absorbed into water to make a dilute HCl solution which is discharged from the bottom of the tail gas separator. Cl₂ is difficult to be absorbed into water in a dilute HCl solution. A large amount of gaseous chlorine escapes from the upper end of the tail gas separator into the chlorine gas dryer in which silica gel or concentrated H₂SO₄ (98%) can be used as the desiccant. The dried Cl₂ and the Cl₂ from the gas source are mixed in the gas mixer and then re-enter the first reactor for the reaction to take place.

A first heat exchanger is mounted at the outlet on the top of the second chlorination reactor. An intermediate receiving drum capable of cooling and heating operation is mounted downstream of the first heat exchanger. It will be beneficial to the collection of volatile materials, and it is necessary for the product to be obtained with a high yield. During the period when the chlorination reaction takes place, 2-methyl pyridine hydrochloride is firstly converted into a large amount of 2-trichloromethyl pyridine and some is further converted into a small amount of 6-chloro-2-trichloromethyl pyridine. 2-trichloro-methyl pyridine has higher volatility and carries some of volatile 6-chloro-2-trichloromethyl pyridine to enter the second reactor and the tail gas recovery unit along with the tail gas stream. This causes the second reactor, the first heat exchanger and the second heat exchanger to be clogged, because 2-trichloromethyl pyridine and 6-chloro-2-trichloromethyl pyridine are solid at a temperature lower than a certain temperature. At this time, 60-80° C. hot water is admitted into the jackets of the second reactor, the first heat exchanger, the intermediate receiving drum and the second heat exchanger to melt the volatile materials clogged in these facilities and pipelines such that the materials in the first heat exchanger and the second reactor are returned to the first reactor and the materials in the second heat exchanger fall into the intermediate receiving drum. The intermediate receiving drum receives colorless, volatile materials, 2-trichloromethyl pyridine and 6-chloro-2-trichloro-methyl pyridine. The molten materials are returned to the first reactor, where they continue to participate in the reaction, or may serve as initiators for the next chlorination reaction of the materials in the reactor. Hence, the material loss will be reduced. In general, during the reaction, the volatile materials received by the intermediate receiving drum account for 5%-30% of the products of the overall reaction. After the reaction is completed, the materials can be melted by heating the intermediate receiving drum to serve as initiators for the next chlorination reaction in the reactor. Prior to material purification, bubbling is carried out in order to draw out the chlorine gas and HCl gas dissolved in the products. The HCl gas and a portion of the Cl₂ gas dissolved in the materials are drawn out under certain temperature conditions by bubbling with vacuum pumping or with compressed air. The purification treatment is carried out until the bubbled materials become neutral. Suitably, the bubbling temperature is 80-100° C. since the materials exhibit very good flowability at this temperature. Alternatively, neutralization is carried out at about 60° C.-80° C. using 5%-10% dilute alkali solution to make the materials neutral followed by the purification.

During the overall reaction, the second reactor has a diameter/height ratio reasonably from 1/12 to 1/26, most suitably 1/18. This ensures that the downwardly flowing 2-methyl pyridine hydrochloride becomes a liquid solution at 20-70° C. The first heat exchanger may be an extension of the second reactor, or may be a single heat exchanger. If it is an extension of the second reactor, the diameter/height ratio is suitably from 1/8 to 1/16, most preferably 1/12. It is feasible to use a heat exchanger, which could collect a large amount of volatile materials and would not be clogged, regardless of the size and shape thereof. The intermediate receiving drum is a vessel that enables both cooling and heating operations. A large amount of volatile materials are collected by the first heat exchanger and enters the first reactor due to the carrying by a large amount of the Cl₂ and HCl during the chlorination reaction. Generally speaking, 5%-30% volatile materials are trapped in the first heat exchanger, the intermediate receiving drum and the second heat exchanger during the chlorination reaction in each reactor to ensure a chlorination reaction of high yield and environmental protection.

The present invention has the beneficial effects as follows. The product of the present invention is useful as a pharmaceutical and agricultural chemical intermediate, and particularly important for fertilizer applications. It can be used as an improved nitrogenous fertilizer for agriculture that causes a delay in the nitration of NH₄⁺ and improves soils and plant nutrition. The preparation method of the present invention possesses features of high selectivity, high yield, better environmental protection, economy and benefit to commercial production.

### BRIEF DESCRIPTION OF THE DRAWINGS

FIG. 1 is a process flow diagram of a continuous reaction according to the present invention;

FIG. 2 is a process flow diagram of an intermittent reaction according to the present invention.

### DETAILED DESCRIPTION OF THE PREFERRED EMBODIMENTS

### Example 1

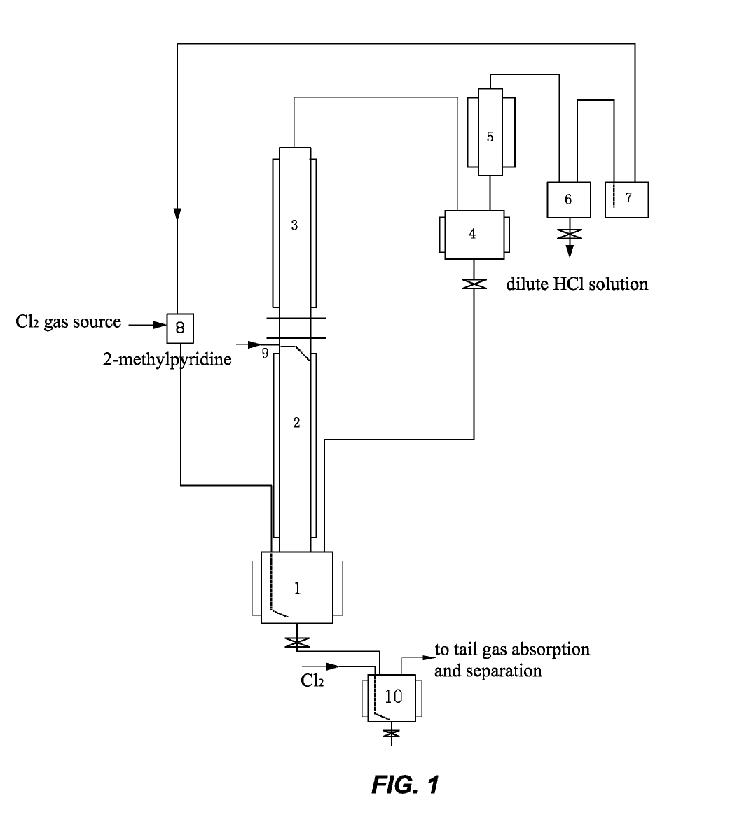

In this example, the method for preparing 6-chloro-2-trichloromethyl pyridine mainly comprises the following steps. Gaseous chlorine and 2-methyl pyridine are used as raw materials, and a continuous reaction is used, as shown in FIG. 1. Firstly, 200 L of liquid 2-methyl pyridine hydrochloride was fed to a first reactor 1 (a 500 L glass-lined reactor) as an initiator. When the temperature rose to 195° C., excess chlorine gas (Cl₂) began to be introduced at 25 kg/hr. 2-methyl pyridine was added dropwise at 2.5 kg/hr along a conduit 9 onto the inner wall of a second reactor 2. The gaseous chlorine reacted with the 2-methyl pyridine hydrochloride to form HCl gas. The HCl gas exited from the first reactor 1 and entered the second reactor 2. It contacted and reacted with a counter-current flow of the 2-methyl pyridine on the inner wall of the second reactor 2 to form 2-methyl pyridine hydrochloride and release heat. The second reactor 2 was temperature controlled using tap water as jacket-cooling water such that heat generated during salt formation was promptly removed from the second reactor 2. Also, the temperature of the second reactor 2 was controlled at 50° C. The formed hydrochloride trickled down along the inner wall of the second reactor 2 due to gravity, dissolved by a large amount of the HCl gas at about 50° C. into 2-methyl pyridine hydrochloride liquid, which flowed along the inner wall of the second reactor 2 into the first reactor 1 and was mixed with the initiator in the first reactor 1 into a liquid system. The liquid system in the first reactor 1 continuously reacted with the continuously introduced excess chlorine gas at 195° C. to initially form 2-trichloromethyl pyridine, which further formed a mixture rich in 6-chloro-2-trichloro-methyl pyridine. After the reaction had taken place for 17 hours, the liquid system in the first reactor 1 reached a stable state. At that time, the liquid system was determined by GC, finding a 6-chloro-2-trichloromethyl pyridine content of 88%, a 2-trichloromethyl pyridine content of 9%, and a 3,6-dichloro-2-trichloromethyl pyridine content of 3%. The bottom valve of the first reactor 1 was opened, and the products in the stable state were continuously withdrawn at a rate of 5.75 kg/hr from the reactor and introduced into the other chlorination reactor 10 connected in series thereto. At that time, 2-methyl pyridine and gaseous chlorine were continuously introduced into the first reactor 1 for chlorination, and the products in the stable state were continuously withdrawn from the outlet at the bottom thereof and entered the chlorination reactor 10 connected in series thereto. Thus, a continuous reaction process was achieved.

A total of 460 kg of materials were received after the chlorination reactor 10 had received the materials withdrawn from the bottom of the first reactor 1 for 80 hours. Chlorine gas was introduced at 5 kg/hr into the chlorination reactor and the reaction began at a reaction temperature of 170° C. under irradiation with ultraviolet light for about 3 hours. Thereafter, the introduction of chlorine gas was stopped and the reactor was cooled down to obtain a total of 474 kg of a mixture. The mixture was determined by GC, finding a 6-chloro-2-trichloromethyl pyridine content of 94.7%, a 2-trichloromethyl pyridine content of 0.4%, a 3,6-dichloro-2-trichloromethyl pyridine content of 3.9%, and a content of a total of the balance, a mixture of 3,5-dichloro-2-trichloromethyl pyridine, 4,6-dichloro-2-trichloromethyl pyridine and the like, of 1%. The mixture was withdrawn from the bottom of the chlorination reactor 10 for purification.

The second reactor 2 is in turn connected to a first heat exchanger 3, an intermediate receiving drum 4, a second heat exchanger 5, a tail gas separator 6, a chlorine gas dryer 7 and a gas mixer 8 at its back end for recovering tail gas generated in the reaction and unreacted chlorine gas, together with a mixture of partially volatile 2-trichloromethyl pyridine and 6-chloro-2-trichloro-methyl pyridine, which is carried by the tail gas and rose and enters the second reactor 2. The first heat exchanger 3 is mounted at the outlet on the top of the second reactor 2. The intermediate receiving drum 4 capable of cooling and heating operation and the second heat exchanger 5 are mounted downstream of the first heat exchanger 3. It will be beneficial to the collection of volatile materials, and it is necessary for the product to be obtained with a high yield. During the period when the chlorination reaction took place, a portion of 2-tri-chloromethyl pyridine and 6-chloro-2-trichloromethyl pyridine frequently rose with the tail gas. The major part was collected back by the second reactor 2 and the first heat exchanger 3 and transmitted to the first reactor 1. A small amount of the uncollected materials entered the intermediate receiving drum 4, and the materials collected back by the second heat exchanger 5 also entered the inter-mediate receiving drum 4. In this manner, the volatile materials can be collected to the greatest extent. Besides, it is effective to prevent the volatile materials which may cause clogging of a tail gas recovery treatment unit from entering it. This will ease the stress of waste disposal, and bring about environmental improvements and high yields. When the first heat exchanger 3, the inter-mediate receiving drum 4 and the second heat exchanger 5 became clogged by the materials, 60-80° C. hot water could be admitted into the jackets of the first heat exchanger 3, the intermediate receiving drum 4 and the second heat exchanger 5 to heat them. At that time, cloggy 6-chloro-2-trichloromethyl pyridine and 2-trichloromethyl pyridine were melted and fell into the first reactor 1 and the intermediate receiving drum 4. The volatile materials in the intermediate receiving drum 4 could be heated, melted and returned to the first reactor 1, where they continued to participate in the reaction, or might serve as initiators for the next reaction in the reactor.

In this example, a total of 52 kg of the colorless, volatile materials were received by the intermediate receiving drum 4 after the reaction had taken place for 80 hours. The materials were determined by GC, finding 13% 2-trichloromethyl pyridine content, 86.5% 6-chloro-2-trichloromethyl pyridine, with the balance, a small amount of 2-methyl pyridine hydrochloride. The materials were heated, melted and entered the first reactor 1, where they continued to participate in the reaction.

A portion of the unreacted chlorine gas, along with a large amount of the tail gas, the HCl gas, flowed through the second reactor 2, the first heat exchanger 3, the intermediate receiving drum 4 and the second heat exchanger 5 into the tail gas separator 6. In the tail gas separator 6, the HCl gas was subjected to film absorption and spray absorption to make a dilute HCl solution which was discharged from the bottom of the tail gas separator 6. Chlorine gas is difficult to be absorbed into water and has very small solubility in a dilute HCl solution. A large amount of wet gaseous chlorine escaped from the upper end of the tail gas separator 6 into the chlorine gas dryer 7 in which silica gel or concentrated H₂SO₄ (98%) could be used as the desiccant. The dried Cl₂ and the Cl₂ from the gas source were mixed in the gas mixer 8 and then re-entered the first reactor 1 for the reaction to take place.

The HCl and Cl₂ dissolved in the mixture rich in 6-chloro-2-trichloromethyl pyridine, which is withdrawn from the bottom of the chlorination reactor 10, must be removed from the mixture before the purification. The HCl is easy to combine with the products to form salts. During the purification, these pyridine hydrochloride salts would be solidified and then further carbonized by heating at a high temperature for a long time into materials difficult to be processed, and the facilities are thus corroded. Hence, the HCl and Cl₂ must be removed from the mixture. According to the present invention, the mixture is heated in a glass-lined vessel in such a manner that the mixture is heated under the condition of 80-90° C. by bubbling with vacuum pumping or with compressed air for about 30 hours. The purification is carried out after it is confirmed that the HCl and Cl₂ in the mixture are removed off.

In this example, said purification was performed by rectification in a plate column. The materials were fed to the middle of a first purification column having 35 theoretical plates, with a rectifying section having 20 theoretical plates and a stripping section having 15 theoretical plates, and operated at a top pressure of 8-10 mmHg, a temperature of 180-190° C. and a reflux ratio of 10:1. The distillate collected at the top temperature of 125-135° C. was a small amount of a low boiling volatile material, 2-trichloromethyl pyridine. The distillate was returned to the first reactor 1 for further chlorination. The materials in the column were continuously pumped and fed to the middle of a second rectification column similarly having 35 theoretical plates. The second rectification column was operated at a top pressure of 20-22 mmHg and a bottom temperature of 200-220° C. The distillate collected at the top temperature of 150-200° C. was very pure 6-chloro-2-trichloromethyl pyridine, which was obtained as a white crystalline solid (m.p. 60-64° C.) after cooling, having a content equal to or greater than 99% as determined by GC. A heavy component in the column was 3,6-dichloro-2-trichloromethyl pyridine useful as an intermediate to produce agricultural herbicides. In this example, 474 kg of a 94.7% 6-chloro-2-trichloromethyl pyridine mixture was rectified to obtain 430 kg of 6-chloro-2-trichloromethyl pyridine having a content of 99% as determined by GC.

### Example 2

The method and basic steps taken in this example are the same as those of Example 1. A continuous reaction is used, as shown in FIG. 1. The weight ratio of Cl₂ to 2-methylpyridine introduced was changed into 4:1, chlorine gas was introduced at 10 kg/hr, and 2-methylpyridine was introduced at a rate of 2.5 kg/hr. The reaction temperature was 195° C. The reaction reached a stable state after 36 hours. The materials in the stable state contained 15% 2-trichloromethyl pyridine, 4.3% 3,6-dichloro-2-tri-chloromethyl pyridine, and 80% 6-chloro-2-trichloro-methyl pyridine. The mixture was introduced into the chlorination reactor 10 for further chlorination. Further chlorination was carried out at a reaction temperature of 170° C. under irradiation with ultraviolet light for a total of 8 hours to obtain a mixture containing 92% 6-chloro-2-trichloromethyl pyridine, 6.5% 3,6-di-chloro-2-trichloromethyl pyridine, and the balance impurities of 1.5%. The content of volatile materials was 85%.

### Example 3

The method and basic steps taken in this example are the same as those of Example 1. A continuous reaction is used, as shown in FIG. 1. The weight ratio of Cl₂ to 2-methylpyridine introduced was changed into 2:1, chlorine gas was introduced at a rate of 5 kg/hr, and 2-methylpyridine was introduced at a rate of 2.5 kg/hr. The materials were carbonized into black tarry matter after the reaction had been carried out for 6 hours. The content of volatile materials was 15%.

### Example 4

The method and basic steps taken in this example are the same as those of Example 1. A continuous reaction is used, as shown in FIG. 1. The weight ratio of Cl₂ to 2-methylpyridine introduced was changed into 20:1. The reaction was carried out at 195° C., and reached a stable state after 17 hours. The materials in the stable state contained 1.5% 2-trichloromethyl pyridine,
3.7% 3,6-di-chloro-2-trichloromethyl pyridine, and 94% 6-chloro-2-trichloromethyl pyridine. The content of volatile materials was 94%.

### Example 5

The method and basic steps taken in this example are the same as those of Example 1. A continuous reaction is used, as shown in FIG. 1. The weight ratio of Cl₂ to 2-methylpyridine introduced was 10:1. The reaction temperature was changed into 140° C. The time taken to reach a stable state was 120 hours. The materials in the stable state contained 79% 2-trichloromethyl pyridine, 17% 6-chloro-2-trichloromethyl pyridine, and 9.5% 3,6-di-chloro-2-trichloromethyl pyridine as determined by GC. The content of volatile materials was 95%.

### Example 6

The method and basic steps taken in this example are the same as those of Example 1. A continuous reaction is used, as shown in FIG. 1. The weight ratio of Cl₂ to 2-methylpyridine introduced was 10:1. The reaction temperature was changed into 230° C. The time taken to reach a stable state was 15 hours. After the stable state was reached, the reactants were continuously withdrawn from the bottom of the first reactor 1 and entered the chlorination reactor 10, where chlorination was carried out at 125° C. under irradiation with ultraviolet light for 10 hours. The resulted mixture contained 1.3% 2-trichloro-methyl pyridine, 3.4% 3,6-dichloro-2-trichloromethyl pyridine, and 94% 6-chloro-2-trichloromethyl pyridine as determined by GC. But the product had a deeper color, containing only 70% volatile materials and the balance of carbonized materials.

### Example 7

The method and basic steps taken in this example are the same as those of Example 1. A continuous reaction is used, as shown in FIG. 1. The weight ratio of Cl₂ to 2-methylpyridine introduced was 10:1. The reaction temperature was 195° C. The reaction reached a stable state after 17 hours. The materials contained 10% 2-trichloro-methyl pyridine, 86% 6-chloro-2-trichloromethyl pyridine, and 4.0% 3,6-dichloro-2-trichloromethyl pyridine. The content of volatile materials was 93%. After the stable state was reached, the reactants were continuously withdrawn from the bottom of the first reactor 1 and entered the chlorination reactor 10, where chlorination was carried out at 230° C. under irradiation with ultraviolet light for 1 hour. The resulted mixture contained 0.8% 2-trichloromethyl pyridine, 94% 6-chloro-2-trichloromethyl pyridine, and 4.7% 3,6-dichloro-2-tri-chloromethyl pyridine as determined by GC. The content of volatile materials was 92.5%.

### Example 8

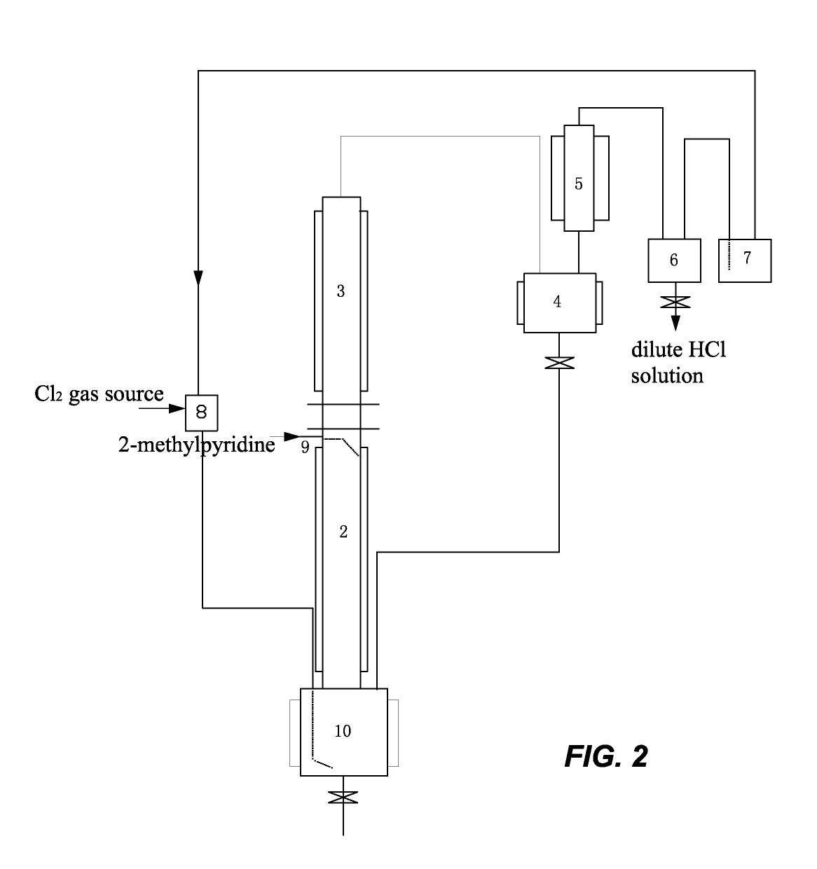

In this example, the method for preparing 6-chloro-2-trichloromethyl pyridine mainly comprises the following steps. Gaseous chlorine and 2-methyl pyridine are used as raw materials. The reaction steps are the same as those of Example 1. Their difference lies in that an intermittent reaction is used. The reaction is stopped and 2-methyl-pyridine is added dropwise into the first reactor when the reaction in the first reactor reaches a stable state. Subsequently, 2-trichloromethyl pyridine is converted into a mixture rich in 6-chloro-2-trichloromethyl pyridine at a reaction temperature of 125° C.-230° C. directly under irradiation with ultraviolet light or under no irradiation, with the continuous introduction of chlorine gas, followed by the purification. As shown in FIG. 2, 200 L of liquid 2-methyl pyridine hydrochloride was firstly fed to a first reactor 1 (a 500 L glass-lined reactor) as an initiator. When the temperature rose to 195° C., excess chlorine gas (Cl₂) began to be introduced at 25 kg/hr. 2-methylpyridine was added dropwise at 2.5 kg/hr along a conduit 9 onto the inner wall of a second reactor 2. After the reaction had taken place for 17 hours, the composition of the reaction mixture, determined by GC, was stable and did not vary any more. At that time, the reaction mixture contained 8.9% 2-trichloromethyl pyridine, 3.1% 3,6-dichloro-2-tri-chloromethyl pyridine, and 88% 6-chloro-2-trichloro-methyl pyridine. At that time, the dropwise addition of 2-methylpyridine was stopped, an ultraviolet lamp was turned on, and the reaction was carried out at 195° C. under irradiation of the ultraviolet lamp and chlorine gas introduced at a rate of 25 kg/hr for about 1.5 hours. At that time, the composition of the reaction mixture was 0.7% 2-trichloromethyl pyridine, 3.4% 3,6-dichloro-2-tri-chloromethyl pyridine, and 94.8% 6-chloro-2-trichloro-methyl pyridine. The content of volatile materials was 94%. The product was discharged from the bottom of the first reactor 1 and subjected to bubbling such that HCl and Cl₂ were removed, followed by the purification with ethanol. White 6-chloro-2-trichloromethyl pyridine was obtained, having a content of 98.3% as determined by GC.

### Example 9

The method and basic steps taken in this example are the same as those of Example 8. An intermittent reaction is used. The initiator was changed into 2-chloromethyl pyridine. The reaction temperature was 195° C. To the 500 L first reactor 1, 65% 2-trichloromethyl pyridine, 15% 2-dichloromethyl pyridine, and 15% 3,6-dichloro-2-tri-chloromethyl pyridine were added. The temperature rose to 195° C., gaseous chlorine was introduced at 25 kg/hr, and 2-methylpyridine was added dropwise at 3 kg/hr. The reaction reached a stable state after 18 hours. At that time, the dropwise addition of 2-methylpyridine was stopped, the temperature was lowered to 150° C., and the reaction was carried out under no irradiation with ultraviolet light and with the introduction of chlorine gas for 14 hours. Thereafter, the product contained 0.9% 2-trichloromethyl pyridine, 4.5% 3,6-dichloro-2-trichloromethyl pyridine, and 93.5% 6-chloro-2-trichloromethyl pyridine as determined by GC.

What is claimed is:

1. A preparation method for 6-chloro-2-trichloro-methyl pyridine, comprising the steps of using gaseous chlorine and 2-methylpyridine as raw materials, firstly, feeding an initiator into a first reactor, continuously introducing excess gaseous chlorine there-into, reacting the gaseous chlorine with the initiator to form HCl gas, the HCl gas rising and entering a second reactor, forming 2-methyl pyridine hydrochloride under controlled temperature conditions in counter-current flow of the raw material, 2-methylpyridine, the hydrochloride re-entering the first reactor with a liquid phase system containing the initiator, reacting with the excess gaseous chlorine to form 2-trichloromethyl pyridine followed by reacting with the continuously introduced excess gaseous chlorine at a certain temperature to form a volatile mixture rich in 6-chloro-2-trichloromethyl pyridine until a stable state is reached, withdrawing the mixture from the bottom of the first reactor, and then purifying the mixture to obtain a 6-chloro-2-trichloromethyl pyridine compound, and the second reactor is connected back to a tail gas recovery unit; the reaction equation is:

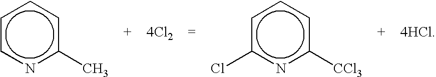

2. The preparation method for 6-chloro-2-trichloro-methyl pyridine as set forth in claim 1, wherein the reaction in said preparation method is a continuous reaction or an intermittent reaction; said purification is performed by rectification in a plate column or recrystallization with ethanol.

3. The preparation method for 6-chloro-2-trichloro-methyl pyridine as set forth in claim 1, wherein the weight ratio of said raw materials of gaseous chlorine to 2-methyl pyridine is from 4:1 to 20:1.

4. The preparation method for 6-chloro-2-trichloro-methyl pyridine as set forth in claim 1, wherein said initiator is a homogeneous solution of liquid 2-methyl pyridine hydrochloride or 2-chloromethyl pyridine.

5. The preparation method for 6-chloro-2-trichloro-methyl pyridine as set forth in claim 1, wherein said certain temperature in the second reactor is the salt forming temperature in the second reactor, which is controlled at 20-70° C.

6. The preparation method for 6-chloro-2-trichloro-methyl pyridine as set forth in claim 1, wherein said certain temperature used in the reaction is the material temperature in the first reactor, which is 140° C.-230° C.

7. The preparation method for 6-chloro-2-trichloro-methyl pyridine as set forth in claim 1 or 2 or 6, wherein when the continuous reaction is used in the preparation method, it comprises directly heating the reactants in the first reactor, the bottom of which is connected in series to the other chlorination reactor, continuously withdrawing the product from the bottom of the first reactor when a stable state is reached, the product entering the other chlorination reactor connected in series thereto, where the reaction is carried out at 125° C.-230° C. under irradiation with ultraviolet light or under no irradiation, with the continuous introduction of chlorine gas and without the introduction of 2-methylpyridine; when the intermittent reaction is used, it comprises stopping the reaction and dropping 2-methylpyridine into the first reactor when the reaction in the first reactor reaches a stable state, and then carrying out the reaction at the reaction temperature from 125° C.-230° C. directly under irradiation with ultraviolet light or under no irradiation, with the continuous introduction of chlorine gas to convert 2-trichloromethyl pyridine into a mixture rich in 6-chloro-2-trichloromethyl pyridine followed by the purification.

8. The preparation method for 6-chloro-2-trichloro-methyl pyridine as set forth in claim 1, wherein said stable state is a state when the proportion of each component in the product does not significantly vary any more after the overall reaction takes place for a certain time.

9. The preparation method for 6-chloro-2-trichloro-methyl pyridine as set forth in claim 1, wherein said tail gas recovery unit in turn comprises the connection of a first heat exchanger, an intermediate receiving drum, a second heat exchanger, a tail gas separator, a gas mixer and a chlorine gas dryer for recovering excess tail gas from the reaction, namely, the gaseous chlorine and HCl gas, together with a mixture of partially volatile 6-chloro-2-trichloromethyl pyridine and 2-trichloromethyl pyridine, which is carried by the tail gas and rises and enters the second reactor, the first heat exchanger, the intermediate receiving drum and the second heat exchanger.

10. The preparation method for 6-chloro-2-trichloro-methyl pyridine as set forth in claim 8, wherein after said mixture of partially volatile 6-chloro-2-trichloromethyl pyridine and 2-trichloromethyl pyridine passes through the second reactor and the first heat exchanger, the major part thereof is collected back to the first reactor, and a small amount of the volatile material again enters the intermediate receiving drum and the second heat exchanger and then is directly recovered into the first reactor through the bottom of the inter-mediate is receiving drum, and the gaseous chlorine and HCl gas are treated in the tail gas separator, subjected to falling film absorption and spray absorption, wherein the HCl gas is absorbed into water to make a dilute HCl solution which is discharged from the bottom of the tail gas separator; a large amount of gaseous chlorine escapes from the upper end of the tail gas separator into the chlorine gas dryer in which silica gel or concentrated H₂SO₄ (98%) is used as the desiccant, and the dried Cl₂ and the Cl₂ from the gas source are mixed in the gas mixer and then re-enter the first reactor for the reaction to take place.
